(19) United States
(12) Patent Application Publication
(10) Pub. No.: US 2013/0197619 A1
(43) Pub. Date: Aug. 1, 2013

(54) INDWELLING DEVICE DELIVERY SYSTEM

(71) Applicant: Terumo Kabushiki Kaisha, Shibuya-ku (JP)

(72) Inventor: Naoki Ishii, Ashigarakami-gun (JP)

(73) Assignee: Terumo Kabushiki Kaisha, Shibuya-ku (JP)

(21) Appl. No.: 13/755,678

(22) Filed: Jan. 31, 2013

Related U.S. Application Data
(60) Provisional application No. 61/592,821, filed on Jan. 31, 2012.

(51) Int. Cl. A61F 2/958

(52) U.S. Cl. CPC A61F 2/958
USPC 623/1.11

(57) ABSTRACT

An indwelling device delivery system including a hollow shaft, a dilatable balloon mounted on a circumference of a distal part of the shaft, and an indwelling device mounted on a circumference of the balloon and expanded by dilatation of the balloon. The balloon has a plurality of protrusions for adhering to the indwelling device and the protrusions are constructed so as to elicit adhesion force based on physical adhesion due to van der Waals force by means of creating more surface area.

### CROSS-REFERENCE TO RELATED APPLICATION

This application is based on U.S. Provisional Application No. 61/592,821 filed on Jan. 31, 2012, the contents of which are incorporated herein by reference.

### BACKGROUND

1. Technical Field The present invention relates to an indwelling device delivery system.

2. Description of the Related Art

A stent is an indwelling device used for improving a narrowed area or an obstructed area generated in a lumen in human body. A stent delivery system has a dilatable balloon that is mounted on the circumference of a distal part of a hollow shaft, and a stent mounted on the outside of the balloon so that the stent can be expanded by the balloon as it expands.

The balloon has protrusions that are engaged with the stent in such a way that any displacement of the stent relative to the balloon and the dislodgement (separation) of the stent and the balloon can be restrained. The protrusions of the balloon are formed by causing a portion of the balloon to extend into a space formed by wire-like members that constitute the stent and having it crimped by the wire-like members (for example, see: Japanese published unexamined Patent Application H08-164210).

However, it causes a problem of making pinholes in the balloon thus during the crimping process and also a problem of weakening the retention force of the stent when it passes through a bending part of a lumen in human body. On the other hand, if the balloon and the stent are secured together by a chemical surface treatment such as the plasma treatment, it also causes a problem of weakening the retention force of the stent when it passes through a bending part of a lumen in human body and a problem that it is difficult to maintain the bonding effect by a chemical interaction in the long term.

### SUMMARY

The present invention is made in order to solve the abovementioned problem associated with the related art, and to provide an indwelling device delivery system that does not weaken the retention force of the indwelling device when the system passes through a bending part of a lumen in human body and keeps the holding effect for a long period of time.

The present invention for achieving said purpose is an indwelling device delivery system including a hollow shaft, a dilatable balloon mounted on a circumference of a distal part of the shaft, and an indwelling device mounted on a circumference of the balloon and expanded by dilatation of the balloon. The balloon has a plurality of protrusions for adhering to the indwelling device and the protrusions are constructed so as to elicit adhesion force based on physical adhesion due to van der Waals force by means of creating more surface area.

According to the present invention, since the adhesion between the balloon and the indwelling device is due to physical adhesion based on van der Waals force, it is possible to prevent the weakening of the retention force of the indwelling device when the system passes through a bending part of a lumen in human body and keeps the holding effect for a long period of time. In other words, the present invention can provide an indwelling device delivery system that does not weaken the retention force of the indwelling device when the system passes through a bending part of a lumen in human body and keeps the holding effect for a long period of time.

In order to make the balloon to be able to disengage from the stent easily when the balloon contracts (deflates), it is preferable that tops of the protrusions that contact the indwelling device are inclined and have directionality. For example, it is possible to incline the top toward a winding direction, or toward a distal side or a proximal side of an axial direction of the shaft.

It is preferable that an area of an adhesive zone where the protrusions are disposed occupies 5% or more of an area of a straight section of the balloon. It is possible to dispose the adhesive zone in a cylindrical shape to surround a circumference of a distal part of the straight section located on a distal side of an axial direction of the shaft, or space a plurality of the adhesive zones from each other on the circumference of the straight section.

If the indwelling device is a stent, it can be used for improving a narrowed area (or obstructed area) developed in a lumen in human body.

The objects, features, and characteristics of this invention other than those set forth above will become apparent from the description given herein below with reference to preferred embodiments illustrated in the accompanying drawings.

### BRIEF DESCRIPTION OF THE DRAWINGS

FIG. 1 is a schematic view of assistance in explaining a stent delivery system according to an embodiment of the present invention.

FIG. 2 is a cross sectional view of assistance in explaining a distal part of the stent delivery system shown in FIG. 1.

FIG. 3 is a cross sectional view taken on line III-III of FIG. 2.

FIG. 4 is a plan view of assistance in explaining a stent shown in FIG. 2.

FIG. 5 is a side view of assistance in explaining a balloon shown in FIG. 2.

FIG. 6 is a cross sectional view of assistance in explaining protrusions arranged in an adhesive zone shown in FIG. 5.

FIG. 7 is a cross sectional view of assistance in explaining the formation of protrusions by means of the blow molding.

FIG. 8 is a cross sectional view of assistance in explaining another example of forming protrusions by means of the blow molding.

FIG. 9 is a side view of assistance in explaining the formation of protrusions by means of the dispensing.

FIG. 10 is a cross sectional view of assistance in explaining the method of using the stent delivery system showing the step of inserting the distal part.

FIG. 11 is a cross sectional view of assistance in explaining the step of expanding the balloon to follow FIG. 10.

FIG. 12 is a cross sectional view of assistance in explaining the step of deflating the balloon to follow FIG. 11.

FIG. 13 is a cross sectional view of assistance in explaining the step of indwelling the stent to follow FIG. 12.

FIG. 14 is a plan view of assistance in explaining a modification 1 according to the embodiment of the present invention.

FIG. 15 is a plan view of assistance in explaining a modification 2 according to the embodiment of the present invention.

FIG. 16 is a plan view of assistance in explaining a modification 3 according to the embodiment of the present invention.

FIG. 17 is a plan view of assistance in explaining a modification 4 according to the embodiment of the present invention.

FIG. 18 is a cross sectional view of assistance in explaining a modification 5 according to the embodiment of the present invention.

FIG. 19 is a cross sectional view of assistance in explaining a modification 6 according to the embodiment of the present invention.

FIG. 20 is a cross sectional view of assistance in explaining a modification 7 according to the embodiment of the present invention.

FIG. 21 is a cross sectional view of assistance in explaining a modification 8 according to the embodiment of the present invention.

FIG. 22 is a cross sectional view of assistance in explaining a modification 9 according to the embodiment of the present invention.

FIG. 23 is a plan view of assistance in explaining a modification 10 according to the embodiment of the present invention.

### DETAILED DESCRIPTION

The embodiment of this invention will be described below with reference to the drawings.

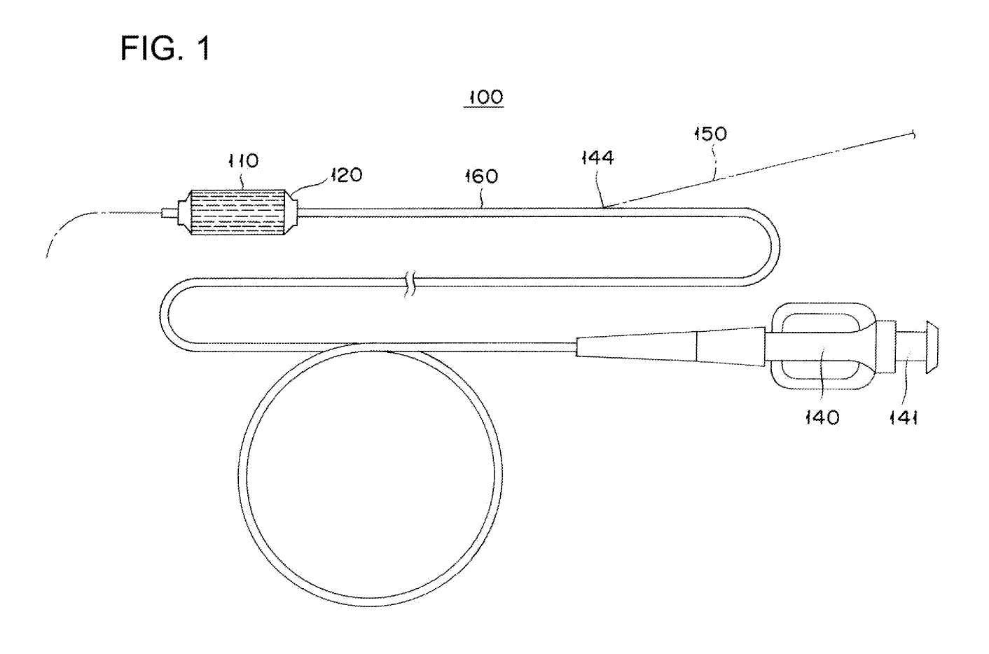

FIG. 1 is a schematic view of assistance in explaining a stent delivery system according to an embodiment of the present invention.

A stent delivery system 100 according to an embodiment of the present invention is of a rapid exchange type with a guide wire 150 passing only through the distal part, is used for improving a narrowed area (or an obstructed area) developed in a lumen in human body, and has a hollow shaft tube 160, a balloon 120 mounted on the circumference of the distal part of the shaft tube 160, a stent 110 mounted on the circumference of the balloon 120, and a hub 140 located at the proximal part of the shaft tube 160 as shown in FIG. 1. Moreover, a guide wire port 144 is provided in the middle of the shaft tube 160. The guide wire port 144 is used for guiding the guide wire 150 through the shaft tube 160 to be projected from the distal part.

A typical lumen in human body is a coronary artery of a human heart, and one of the objects of improving the narrowed area is to prevent restenosis after a percutaneous transluminal coronary angioplasty (PTCA). The stent delivery system 100 can be applied not only to narrowed areas developed in coronary arteries of heart but also to narrowed areas developed in other lumens such as a blood vessel, bile duct, trachea, esophagus, urethra, etc.

The stent 110 is an indwelling device that supports a lumen by closely contacting the inside of a narrowed area and being left inside the lumen, and is adapted to be able to expand. The balloon 120 is dilatable and adapted to expand the stent 110 which is located outside thereof, thus expanding its diameter.

The hub 140 has a port 141. The port 141 is used, for example, to introduce or discharge a pressurized fluid for the purpose of expanding the balloon 120. The pressurized fluid can be a liquid such as physiological saline or angiographic agent.

Next, the distal part of the stent delivery system 100 will be described in detail.

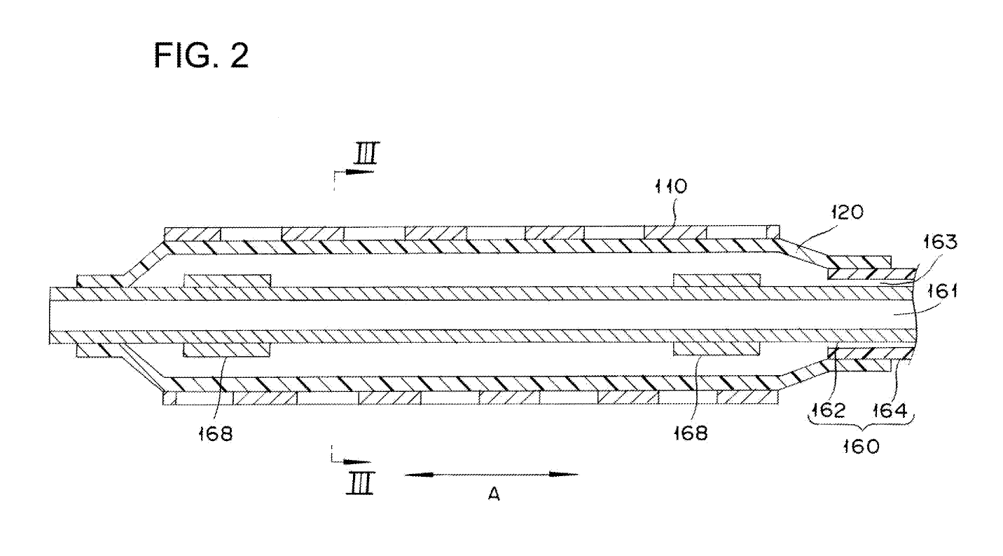

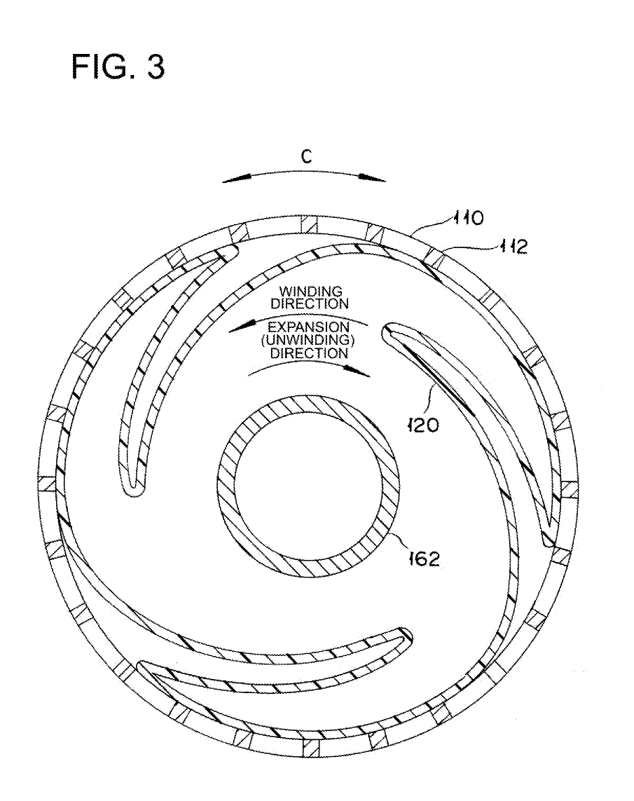

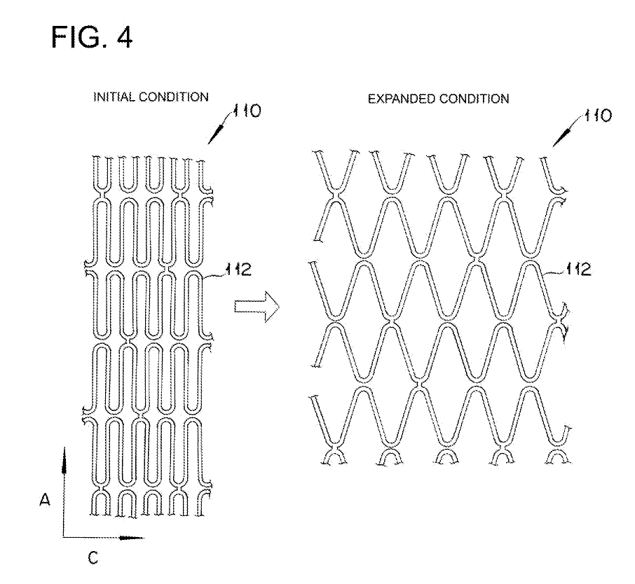

FIG. 2 is a cross sectional view of assistance in explaining the distal part of the stent delivery system shown in FIG. 1, FIG. 3 is a cross sectional view taken on line III-III of FIG. 2, and FIG. 4 is a plan view of assistance in explaining the stent shown in FIG. 2.

The stent 110 is, as shown in FIG. 4, formed by disposing annular parts composed of undulate wire-like members 112 which extend in a circumferential direction C and which can expand and contract in a radial direction, in parallel in an axial direction A and joining them together. Moreover, the stent 110 is not limited to the above-mentioned constitution.

The stent 110 is made of a biocompatible material. The biocompatible materials include nickel-titanium alloy, cobalt-chromium alloy, stainless steel, iron, titanium, aluminum, tin, and zinc-tungsten alloy, for example.

The balloon 120 is dilatable and is mounted on the circumference of the distal part of the shaft tube 160 in a folded condition (or deflated condition) by being wound around as shown in FIG. 3, and is unwound when it is expanded. Since the balloon 120 is located inside the stent 110, the wire-like members 112 of the stent 110 are expanded due to the dilatation of the balloon 120. For example, the area (MSA) of the balloon 120 which can come in contact with the stent 110 is 60% when it is in the folded condition, and occupies approximately 20% when it is expanded. Moreover, the balloon 120 does not have to be limited to the folded embodiment.

The material to be used for the balloon 120 is preferably a material having flexibility such as polyolefin, polyvinyl chloride, polyamide including nylon, polyamide elastomer, polyurethane, polyester such as polyethylene terephthalate (PET), polyarylene sulfide such as polyphenylene sulfide, silicone rubber, and latex rubber. The polyolefin includes, for example, polyethylene, polypropylene, ethylene-propylene copolymer, ethylene-vinyl acetate copolymer, and cross-linked ethylene-vinyl acetate copolymer.

The shaft tube 160 has, as shown in FIG. 2, an inner tube 162, and an outer tube 164 into which the inner tube 162 is inserted. The inner tube 162 is communicating with a guide wire port 144, and extends out to the distal end through the balloon 120. Therefore, the guide wire inserted into the guide wire port 144 can be projected from the distal end of the stent delivery system 100. In other words, the inside of the inner tube 162 constitutes a lumen 161 for the guide wire.

The inner tube 162 is attached with coil-shaped markers 168 that align with both ends of the stent 110. The markers 168 are made of a radiopaque material in order to provide clear images under a radioscopic view, so that they can be used for easily identifying the positions of the stent 110 (the distal part of the stent delivery system 100). The radiopaque material can be, for example, platinum, gold, tungsten, iridium, and their alloys.

The outer tube 164 is placed on the outside of the inner tube 162, and the lumen 163 consisting of a space between the inner circumference of the outer tube 164 and the outer circumference of the inner tube 162 is communicating with the port 141 of the hub 140. The balloon 120 is fixed on the outer circumference of the distal part of the outer tube 164 in a fluid-tight manner, and the inside of the balloon 120 is communicating with the lumen 163. Therefore, the pressurized fluid supplied from the port 141 is introduced into the inside of the lumen 120 via the lumen 163, so as to be able to expand the balloon 120. The method of fixing the balloon 120 to the outer circumference of the distal part of the outer tube 164 is not specified particularly and either adhesives or thermal fusion means can be used.

The outer tube 164 should preferably be made of a material with flexibility, for example, polyolefins such as polyethylene, polypropylene, polybutene, ethylene-propylene copolymer, ethylene-vinyl acetate copolymer, ionomer, or any mixture of more than two kinds of them, plasticized polyvinyl chloride resin, polyamide, polyamide elastomer, polyester, polyester elastomer, polyurethane, thermoplastic resins such as fluorocarbon resin, silicone rubber, and latex rubber.

The material to be used for the inner tube 162 can be the same as the material for the outer tube 164, or metal materials. The metal materials can be, for example, stainless steel, extensible stainless alloy, and Ni—Ti alloy.

The material to be used for the hub 140 (see FIG. 1) can be, for example, thermoplastic resins such as polycarbonate, polyamide, polysulfone, Polyarylate, and methacrylate-butylene-styrene copolymer.

Next, the balloon will be discussed about in detail.

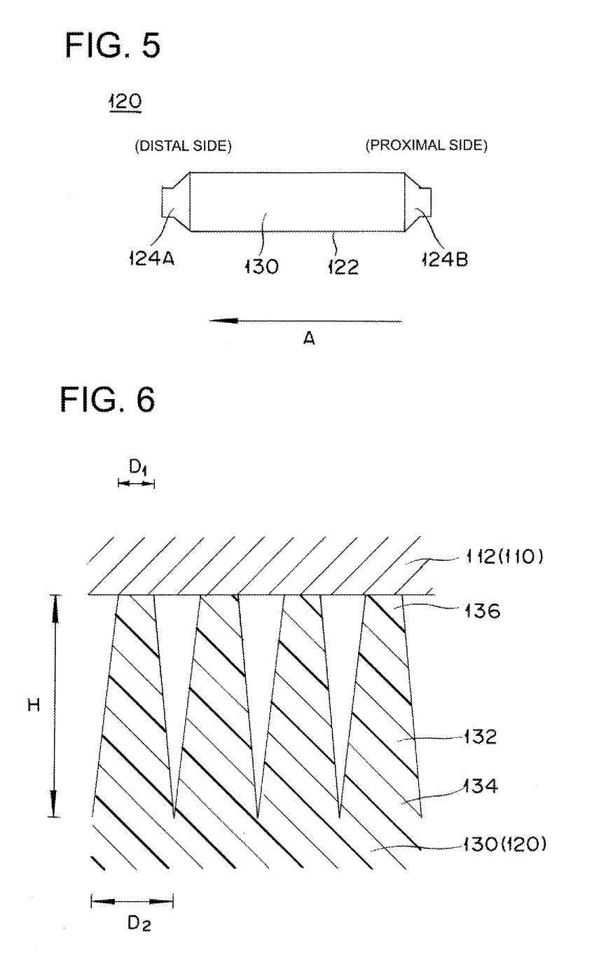

FIG. 5 is a side view of assistance in explaining the balloon shown in FIG. 2, and FIG. 6 is a cross sectional view of assistance in explaining protrusions arranged in an adhesive zone shown in FIG. 5.

The balloon 120 has a straight section 122 and radially shrinking sections 124A, 124B. The straight section 122 is, for example, formed as a cylinder with an outer diameter of approximately 3 mm, and has an adhesive zone 130 arranged all over the surface. The radially shrinking sections 124A, 124B have a diameter smaller than that of the straight section 122 and located on both ends of the straight section 122 in the axial direction A, wherein the radially shrinking section 124A is on the distal side, and the radially shrinking section 124B is on the proximal side.

The adhesive zone 130 has a plurality of minute truncated cone-shaped protrusions 132 of nanometer order or micrometer order is provided as shown in FIG. 6. The protrusions 132 are constructed so as to elicit adhesion force based on van der Waals force by means of creating more surface area of the part that contacts the wire-like members 112 of the stent 110. For example, the diameter D₂ of the base 134 of the protrusions 132 and the diameter D₁ of the top 136 of the protrusions 132 that contacts the stent 110 is 5 nm to 10 pm and the height H of the protrusions 132 is 1 μm to 30 μm and the formation density is higher than one per μm² and thus the minute protrusions 312 are densely formed. For example, a microscopic fibrous structure which is often observed on the back side of a foot of a gecko is generally known as the structure that adheres using the van der Waals force.

As the protrusions 312 elicit physical adhesion force due to the van der Waals force that works on the contacting with wire-like members 112 (stent 110), it sustains an adhesive condition without requiring any chemical surface treatment and the like, eliminates the need of any preparation for the chemical surface treatment, does not cause any deterioration of the retention force even when the system passes through a bending part, and maintains the effect for a long period of time. In other words, as the adhesion force between the balloon 120 and the stent 110 is based on a physical adhesion force due to the van der Waals force, the retention force of the stent 110 does not drop even when it passes through a bending part (e.g., aortic aneurysm) of a lumen in human body and maintains the holding effect for a longer period of time.

The protrusion 132 does not have to be to be in a truncated cone shape, but rather can be, for example, in a columnar shape, or in a pillar-like shape with a polygonal cross section or with a cross section at the top larger than that of the base.

Next, the method of producing the protrusions 132 will be described below.

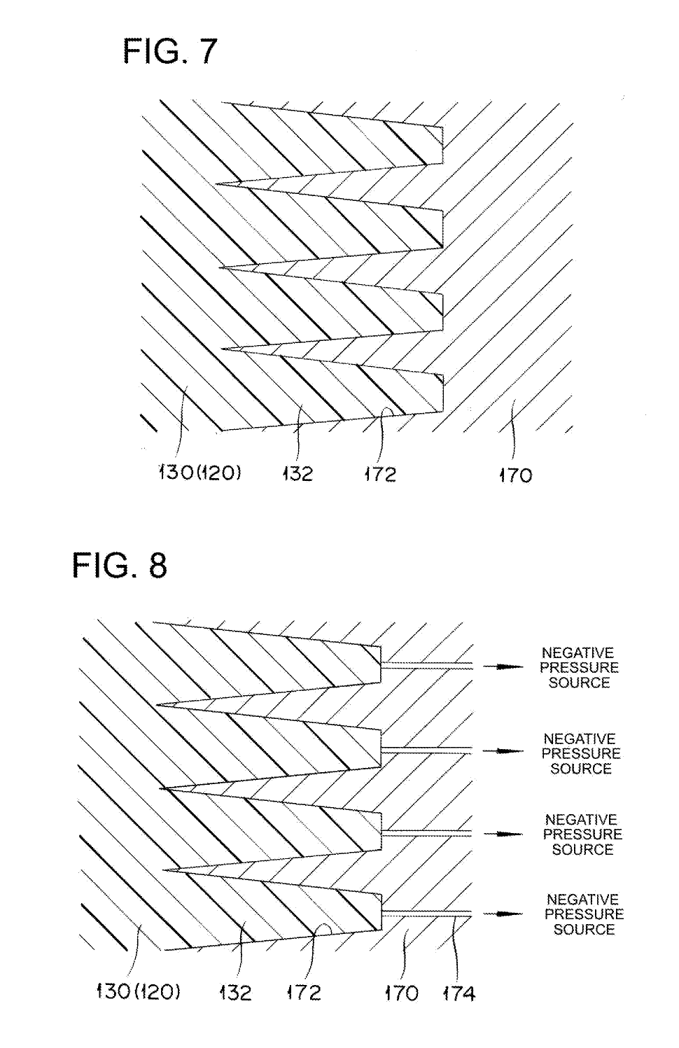

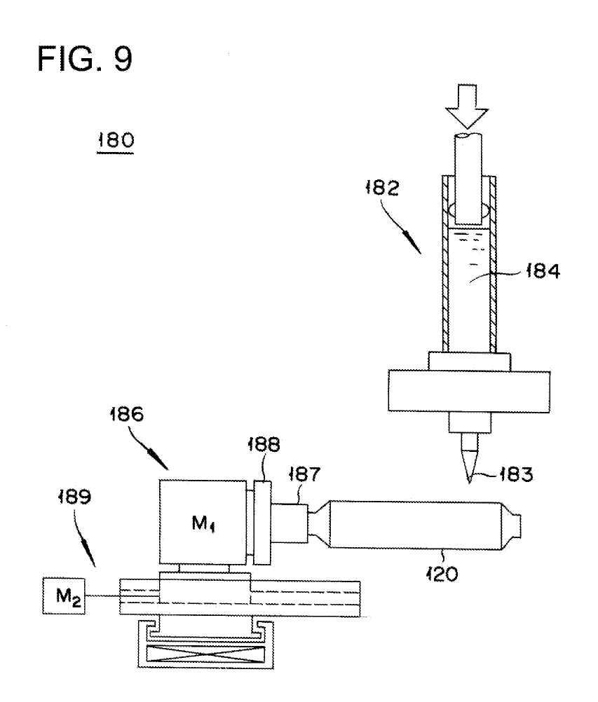

FIG. 7 is a cross sectional view of assistance in explaining the formation of protrusions by means of the blow molding, FIG. 8 is a cross sectional view of assistance in explaining another example of forming protrusions by means of the blow molding, and FIG. 9 is a side view of assistance in explaining the formation of protrusions by means of the dispensing.

The method of forming the protrusions 132 is not specified particularly, but rather any balloon forming or coating can be applied. For example, in case the balloon is formed by means of the blow molding, it is possible to simultaneously form the balloon 120 and the protrusions 132 in forming the balloon 120 using a balloon mold if depressed portions 172 that correspond to the protrusions 132 are provided in the cavity of a mold 170 for forming the balloon 120 as shown in FIG. 7. It is also possible to expedite the flow of the balloon material (balloon 120) into the depressed portions 172 by providing in the mold 170 fine through-holes 174 that connects the depressed portions 172 with the negative pressure source as shown in FIG. 8.

The coating can be made by means of dispensing and ink jet. For example, if the dispensing is applied for coating as shown in FIG. 9, it is possible to form the protrusions 132 consisting of a nonvolatile composition by intermittently discharging a solution 184 containing a solvent and the nonvolatile composition that is to constitute the protrusions 132 against the adhesive zone 130 of the balloon 120 from a nozzle 183 of a dispenser 182 in accordance with the shape and the arrangement pattern of the protrusions 132, and then causing the solvent to volatilize from the solution 184.

The balloon 120 is attached to a holding fixture 186 via a mandrel 187 and a chuck 188 in an expanded condition, and driven by a motor M₁. The holding fixture 186 is configured in such a manner as to be able to move in two axis directions (two directions crossing each other perpendicularly relative to the axial direction of the dispenser 182) by a moving means 189 having a motor M₂ as a driving source. The inner diameter of the distal part of the nozzle 183 can be suitably set according to the shape and size of the protrusions 132.

It is also possible to use the femtosecond laser to form the protrusions 132. The femtosecond laser is a laser in which the duration of a laser pulse is extremely shortened, as short as the order of femtosecond (a 10⁻¹⁵ of a second), so that the energy (laser) is irradiated in an extremely short period of time, so that the machining of the workpiece materials can be completed before the heat is transferred to points other than the irradiated point, thus making it possible to conduct a very fine machining. Therefore, it is possible to form a micro structure surface having the protrusions 132 by irradiating the balloon 120 with a femtosecond laser in a scanning manner along the shape and arrangement pattern of the protrusions 132.

When a workpiece is irradiated with a linearly polarized femotosecond laser, it shows a characteristic of self-organizationally forming a grating-like structure having a cycle equivalent to the wavelength of the laser based on the interference of the incident light and the scattering light or plasma wave along the surface of the workpiece. Accordingly, using this characteristic, it is possible to self-organizationally form a micro structured surface having the protrusions 132 over the entire region where it is irradiated with the linearly polarized femtosecond laser by scanning with the laser in an overlapping manner.

The method of forming the protrusions 132 can be suitably chosen from such means as nanoimprint, laser, soft lithography, machining (e.g., micro diamond turning tool), etc. in addition to the aforementioned molding, coating and femtosecond laser, depending on the shapes, dimensions, materials, etc. of the protrusions 132.

Next, the method of using the stent delivery system 100 will be described in detail.

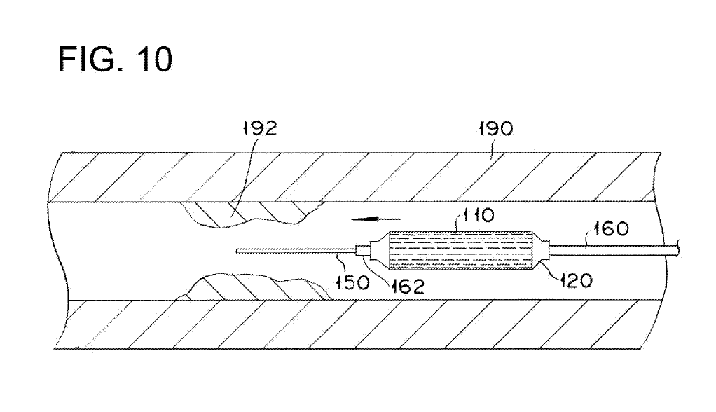

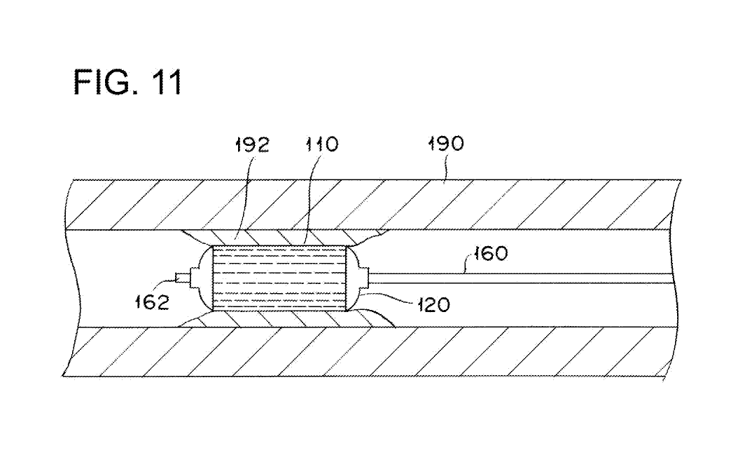

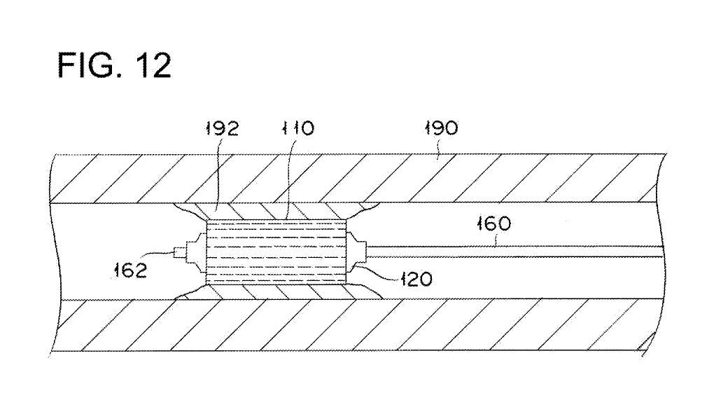

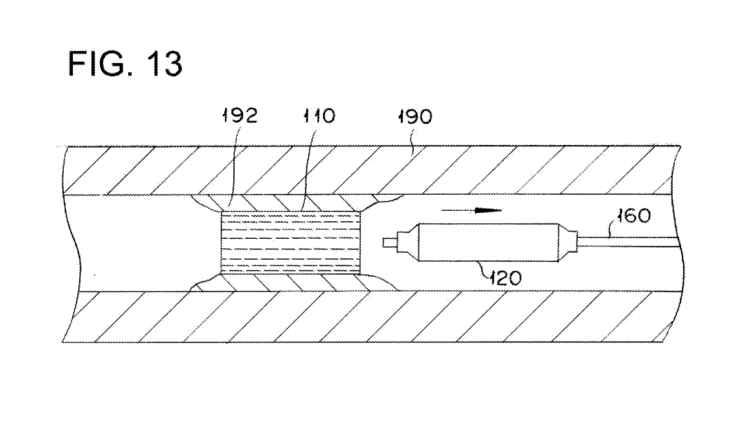

FIG. 10 is a cross sectional view of assistance in explaining the method of using the stent delivery system showing the step of inserting the distal part, FIG. 11 is a cross sectional view of assistance in explaining the step of expanding the balloon to follow FIG. 10, FIG. 12 is a cross sectional view of assistance in explaining the step of deflating the balloon to follow FIG. 11, and FIG. 13 is a cross sectional view of assistance in explaining the step of indwelling the stent to follow FIG. 12.

The method of using the stent delivery system 100 generally includes a distal part insertion step, a balloon dilatation step, a balloon deflation step and a stent deployment step.

In the distal part insertion step, the distal part of the shaft tube 160 (inner tube 162) is inserted into a patient's lumen 190 and moved toward a narrowed area 192 which is the target location while preceding the guide wire 150 as shown in FIG. 10. In this case, since the protrusions 312 of the adhesive zone 130 of the balloon 120 elicit physical adhesion force due to the van der Waals force that works on the contacting with wire-like members 112 (stent 110), the retention force of the stent 110 does not drop even when it passes through a bending part (e.g., aortic aneurysm) of s lumen in human body and maintains the holding effect for a longer period of time. The guide wire 150 is inserted into the inner tube 162 via the guide wire port 144 and projects from the distal end of the inner tube 162.

In the balloon dilatation step, as shown in FIG. 11, as the stent 110 is positioned at the narrowed area 192, which is the target location, a pressurized fluid is supplied via the port 141, and then introduced to the inside of the balloon 120 via the lumen 163 (see FIG. 2) to inflate (expand) the balloon 120. As a result, it also causes the stent 110, which is located on the outer circumference of the balloon 120, to expand to contact closely the surface of the narrowed area 192. The positioning of the stent 110 can be accurately, quickly and easily conducted by visually checking the positions of the markers 168 (FIG. 2) aligned with both ends of the stent 110 with the help of X-ray radiation.

In the balloon deflation step, as shown in FIG. 12, the pressurized fluid is discharged from the port 141 via the lumen 163 to allow the balloon 120 to contract (deflate). At this time, the stent 110 is plastically deformed, and the physical adhesion between the balloon 120 and the stent 110 by means of the protrusions can easily withdraw from the sent 110 when the balloon 120 contracts (deflates), so that the stent 110 does not accompany the contraction of the balloon 120. Thus, the stent 110 is separated from the balloon 120.

In the stent deployment step, as shown in FIG. 13, the distal part of the shaft tube 160, from which the stent 110 has separated, retracts and is removed from the lumen 190.

Next, modifications 1 through 10 of the present invention will be sequentially described in the following.

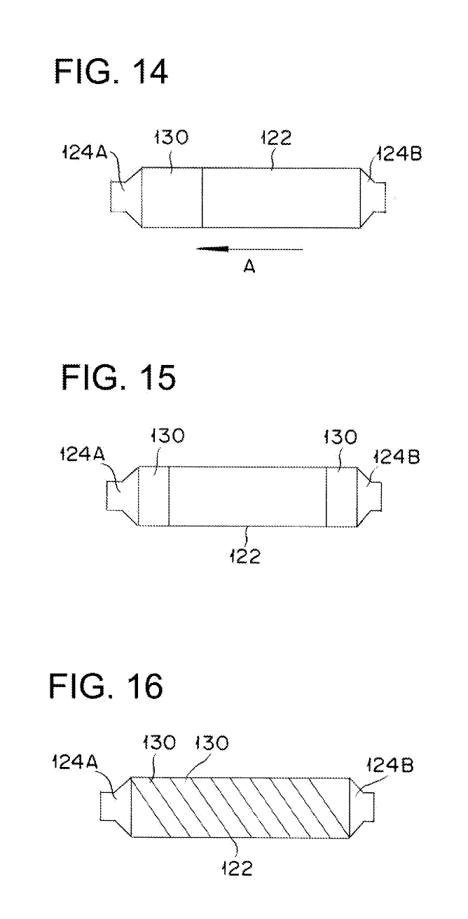

FIGS. 14 through 17 are plan views of assistance in explaining the modifications 1 through 4 according to the embodiment of the present invention.

The adhesive zone 130 of the balloon 120 does not have to be formed to extend over the entire surface of the straight section of the balloon 120, but rather can be placed only partially as needed.

For example, the adhesive zone 130 can be placed on the distal side of the straight section 122 as shown in FIG. 14 on ground that the stent's distal side is impinged against the narrowed area when it passes through the narrowed area. In this case, the length of the adhesive zone 130 relative to the axial direction A should be longer than 5%, or preferably be longer than 10%, or more preferably be longer than 25% of the length of the straight section 122.

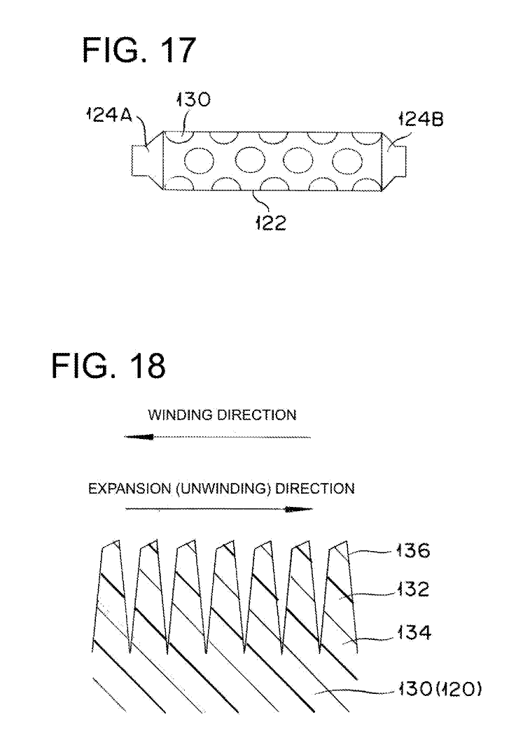

The adhesive zone 130 can be constituted for a plurality of regions placed separately (non-continuously) as needed. For example, the adhesive zone 130 can be divided into two regions placed in the vicinities of both ends of the straight section 122 as shown in FIG. 15, or the adhesive zone 130 can be composed of a plurality of ring-shaped regions disposed equal distance apart along the axial direction A as shown in FIG. 16, or the adhesive zone 130 can be composed of a plurality of circular regions disposed equal distance apart from each other on the outer circumference of the straight section 122 as shown in FIG. 17. In this case, the entire area of the adhesive zone 130 should be more than 5%, or preferably be more than 10%, or more preferably be more than 25% of the area of the straight section 122.

FIGS. 18 through 20 are cross sectional views of assistance in explaining the modifications 5 through 7 according to the embodiment of the present invention.

The tops 136 of the protrusions 132 in the adhesive zone 130 that come in contact with the stent 110 do not have to be flat, but rather can have directionality. For example, in case the top 136 is inclined toward the winding direction relative to the circumferential direction as shown in FIG. 18, the adhesion force weakens as the contact area between the top 136 and the stent 110 reduces when the balloon 120 expands at the balloon dilatation step, while the balloon 120 becomes easily separating from the stent 110 as the diameter of the balloon 120 is reduced with the top 136 sliding on the internal surface of the stent in the winding direction when the balloon 120 deflates at the balloon deflation step.

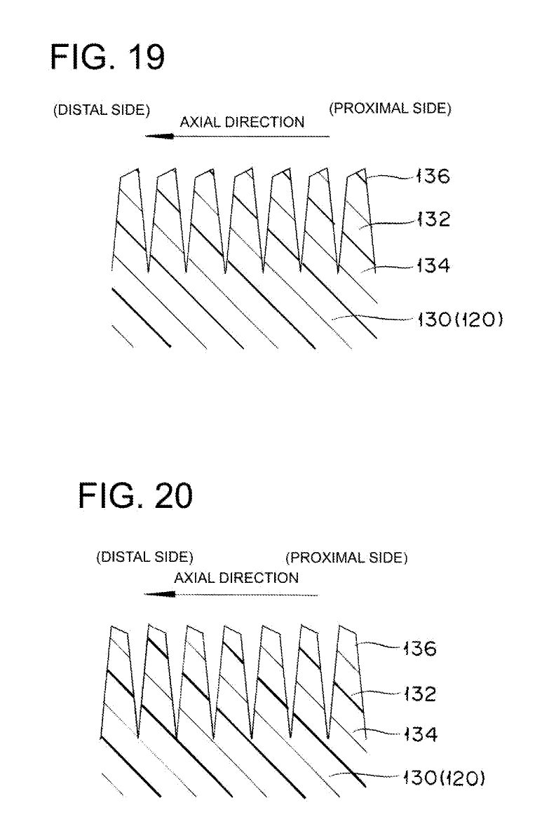

Moreover, if the top 136 is inclined toward the distal side with reference to the axial direction, for example, as shown in FIG. 19, the balloon 120 becomes easier to be separated from the stent 110 by pulling (moving) the balloon 120 toward the proximal side during the deflation of the balloon 120, while, if the top 136 is inclined toward the proximal side as shown in FIG. 20, the balloon 120 becomes easier to be separated from the stent 110 by pushing (moving) the balloon 120 toward the distal side.

The angle of inclination is not specified particularly and can be set suitably depending on the shape, dimension, material, etc. of the protrusions 132.

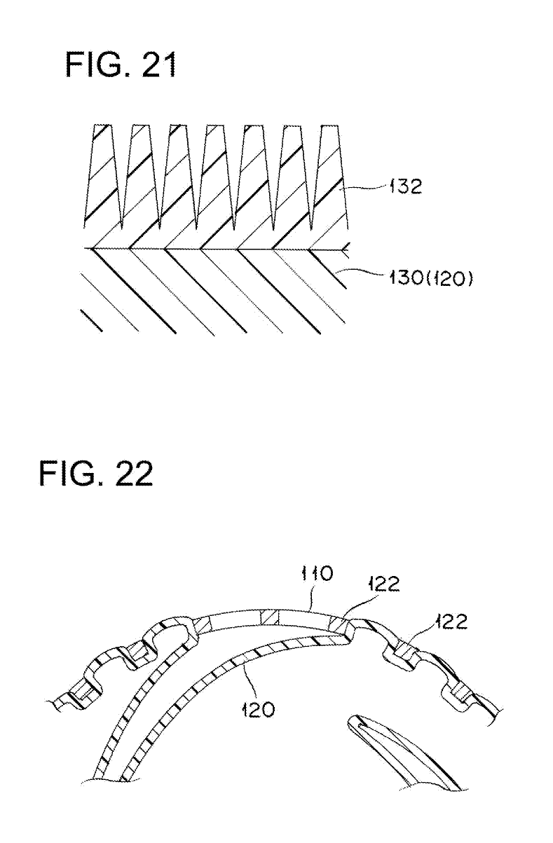

FIG. 21 is a cross sectional view of assistance in explaining the modification 8 according to the embodiment of the present invention.

The protrusions 132 do not have to be made of the same material as that of the balloon 120 but can be made of a different material. For example, if a coating is to be applied, the protrusions 132 can be formed from a different material (nonvolatile composition) by coating the balloon 120 with coating compositions containing the material (nonvolatile composition) different from that of the balloon 120 and a solvent in accordance with the shape and arrangement pattern of the protrusions 132, and then causing the solvent to volatilize from the coat.

FIG. 22 is a cross sectional view of assistance in explaining the modification 9 according to the embodiment of the present invention.

The stent 110 can be engaged by crimping the balloon 120 as shown in FIG. 22 as needed. In this case, it is easy for the balloon 120 to secure a sufficient stent retention force (fixing strength) as it has the adhesive zone 130 and then a small force is sufficient to tuck (crimp) to engage the balloon 120 by the stent 110, so that the occurrence of pinholes due to the crimping force can be prevented.

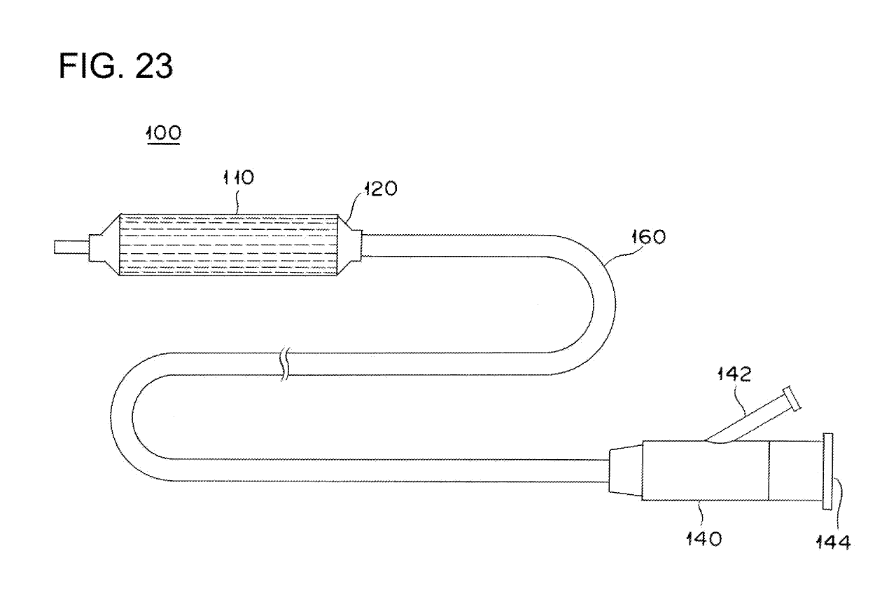

FIG. 23 is a plan view of assistance in explaining the modification 10 according to the embodiment of the present invention.

The stent delivery system 100 does not have to be limited to the rapid exchange type, but also it can be the over-the-wire (OTW) type system as shown in FIG. 23. Since the guide wire passes through from the distal end to the proximal end, the guide wire replacement and operation are easier in this case. The symbol 142 represents the injection port used for introducing and discharging the pressurized fluid for expanding the balloon 120, and the symbol 144 represents the guide wire port for inserting the guide wire to project it from the distal part through the shaft tube 160.

As can be seen in the above, since the adhesion between the balloon and the stent (indwelling device) is due to physical adhesion based on van der Waals force in the present embodiment, it is possible to prevent the weakening of the retention force of the stent when the system passes through a bending part of a lumen in human body and keeps the holding effect for a long period of time. In other words, the present invention can provide a stent delivery system that does not weaken the retention force of the stent when the system passes through a bending part of a lumen in human body and keeps the holding effect for a long period of time.

The present invention shall not be construed to be limited by the embodiment described above but rather it can be modified in a various way within the claims. For example, the indwelling device does not have to be limited to a stent but is applicable for a pessary.

What is claimed is:

1. An indwelling device delivery system comprising:
a hollow shaft;
a dilatable balloon mounted on a circumference of a distal part of said shaft; and
an indwelling device mounted on a circumference of said balloon and expanded by dilatation of said balloon, wherein
said balloon has a plurality of protrusions for adhering to said indwelling device, and said protrusions are constructed so as to elicit adhesion force based on physical adhesion due to van der Waals force by means of creating more surface area.

2. The indwelling device delivery system as claimed in claim 1, wherein tops of said protrusions that contact said indwelling device are inclined and have directionality.

3. The indwelling device delivery system as claimed in claim 2, wherein said balloon is mounted on the circumference of the distal part of said shaft in a wound state and said tops are inclined in a winding direction.

4. The indwelling device delivery system as claimed in claim 2, wherein said tops are inclined toward a distal side or a proximal side of an axial direction of said shaft.

5. The indwelling device delivery system as claimed in claim 1, wherein said balloon has a cylindrical straight section and a radially shrinking section having a diameter smaller than a diameter of said straight section and said protrusions are disposed on said straight section and an area of an adhesive zone where said protrusions are disposed occupies 5% or more of an area of said straight section.

6. The indwelling device delivery system as claimed in claim 5, wherein said adhesive zone is disposed in a cylindrical shape to surround a circumference of a distal part of the straight section located on a distal side of an axial direction of said shaft.

7. The indwelling device delivery system as claimed in claim 5, wherein there are provided a plurality of said adhesive zones spaced from each other on the circumference of said straight section.

8. The indwelling device delivery system as claimed in claim 1, wherein said indwelling device is a stent.
